(19) United States
(12) Patent Application Publication
(10) Pub. No.: US 2011/0071516 A1
(43) Pub. Date: Mar. 24, 2011

(54) System and Method for Controlling Electrosurgical Output

(75) Inventor: William N. Gregg, Superior, CO (US)

(73) Assignee: TYCO Healthcare Group LP

(21) Appl. No.: 12/566,233

(22) Filed: Sep. 24, 2009

(51) Int. Cl. A61B 18/12

(52) U.S. Cl. 606/34

(57) ABSTRACT

An electrosurgical generator for supplying electrosurgical energy to tissue includes sensor circuitry configured to measure a voltage phase and a current phase through tissue and a processing device configured to compare the measured voltage and current phase to generate a real power component. The electrosurgical generator also includes a controller configured to regulate output of the electrosurgical generator based on the real power component and/or a predetermined imaginary impedance of tissue.

### BACKGROUND

1. Technical Field

The present disclosure relates to electrosurgical apparatuses, systems and methods. More particularly, the present disclosure is directed to electrosurgical systems and methods for monitoring electrosurgical procedures based on real power.

2. Background of Related Art

Energy-based tissue treatment is well known in the art. Various types of energy (e.g., electrical, ohmic, resistive, ultrasonic, microwave, cryogenic, laser, etc.) are applied to tissue to achieve a desired result. Electrosurgery involves application of high radio frequency electrical current to a surgical site to cut, ablate, coagulate or seal tissue. In monopolar electrosurgery, a source or active electrode delivers radio frequency energy from the electrosurgical generator to the tissue and a return electrode carries the current back to the generator. In monopolar electrosurgery, the source electrode is typically part of the surgical instrument held by the surgeon that is applied to the tissue. A patient return electrode is placed remotely from the active electrode to carry the current back to the generator.

Ablation is most commonly a monopolar procedure that is particularly useful in the field of cancer treatment, where one or more RF ablation needle electrodes that (usually of elongated cylindrical geometry) are inserted into a living body. A typical form of such needle electrodes incorporates an insulated sheath disposed over an exposed (uninsulated) tip. When the RF energy is provided between the return electrode and the inserted ablation electrode, RF current flows from the needle electrode through the body. Typically, the current density is very high near the tip of the needle electrode, which tends to heat and destroy surrounding issue.

In bipolar electrosurgery, one of the electrodes of the hand-held instrument functions as the active electrode and the other as the return electrode. The return electrode is placed in close proximity to the active electrode such that an electrical circuit is formed between the two electrodes (e.g., electrosurgical forceps). In this manner, the applied electrical current is limited to the body tissue positioned between the electrodes. When the electrodes are sufficiently separated from one another, the electrical circuit is open and thus inadvertent contact with body tissue with either of the separated electrodes prevents the flow of current.

Bipolar electrosurgical techniques and instruments can be used to coagulate blood vessels or tissue, e.g., soft tissue structures, such as lung, brain and intestine. A surgeon can either cauterize, coagulate/desiccate and/or simply reduce or slow bleeding, by controlling the intensity, frequency and duration of the electrosurgical energy applied between the electrodes and through the tissue. In order to achieve one of these desired surgical effects without causing unwanted charring of tissue at the surgical site or causing collateral damage to adjacent tissue, e.g., thermal spread, it is necessary to control the output from the electrosurgical generator, e.g., power, waveform, voltage, current, pulse rate, etc.

It is known that measuring the electrical impedance and changes thereof across the tissue at the surgical site provides a good indication of the state of desiccation or drying of the tissue, e.g., as the tissue dries or loses moisture, the impedance across the tissue rises. This observation has been utilized in some electrosurgical generators to regulate the electrosurgical power based on measured tissue impedance.

### SUMMARY

According to one embodiment of the present disclosure, an electrosurgical generator for supplying electrosurgical energy to tissue includes sensor circuitry configured to measure a voltage phase and a current phase through tissue and a processing device configured to compare the measured voltage and current phase to generate a real power component. The electrosurgical generator also includes a controller configured to regulate output of the electrosurgical generator based on the real power component and/or a predetermined imaginary impedance of tissue.

According to another embodiment of the present disclosure, a method for supplying electrosurgical energy to tissue includes the steps of measuring a voltage phase and a current phase through tissue and comparing the measured voltage phase and the measured current phase to generate a real power component. The method also includes the step of regulating output of an electrosurgical generator based on the real power component and/or a predetermined range between a complex impedance of tissue and a real impedance of tissue.

According to another embodiment of the present disclosure, a method for supplying electrosurgical energy to tissue includes the steps of measuring a voltage phase and a current phase through tissue and calculating a difference between the voltage phase and the current phase. The method also includes the step of calculating a real power component of the electrosurgical energy based on one or more of the voltage phase, the current phase, and a difference between the voltage and current phases. The method also includes the steps of regulating output of the electrosurgical generator based on the real power component and adjusting the output of the electrosurgical generator to a pre-determined safeguard level based on a detected fault condition.

### BRIEF DESCRIPTION OF THE DRAWINGS

Various embodiments of the present disclosure are described herein with reference to the drawings wherein:

FIG. 1A is a schematic block diagram of a monopolar electrosurgical system according to one embodiment of the present disclosure;

FIG. 1B is a schematic block diagram of a bipolar electrosurgical system according to one embodiment of the present disclosure;

FIG. 2 is a schematic block diagram of a generator according to an embodiment of the present disclosure; and

FIG. 3 is a flow chart of a method according to an embodiment of the present disclosure.

### DETAILED DESCRIPTION

Particular embodiments of the present disclosure are described hereinbelow with reference to the accompanying drawings. In the following description, well-known functions or constructions are not described in detail to avoid obscuring the present disclosure in unnecessary detail.

The generator according to the present disclosure can perform monopolar and bipolar electrosurgical procedures, including vessel sealing procedures. The generator may include a plurality of outputs for interfacing with various electrosurgical instruments (e.g., a monopolar active electrode, return electrode, bipolar electrosurgical forceps, footswitch, etc.). Further, the generator includes electronic circuitry configured for generating radio frequency power specifically suited for various electrosurgical modes (e.g., cutting, blending, division, etc.) and procedures (e.g., monopolar, bipolar, vessel sealing).

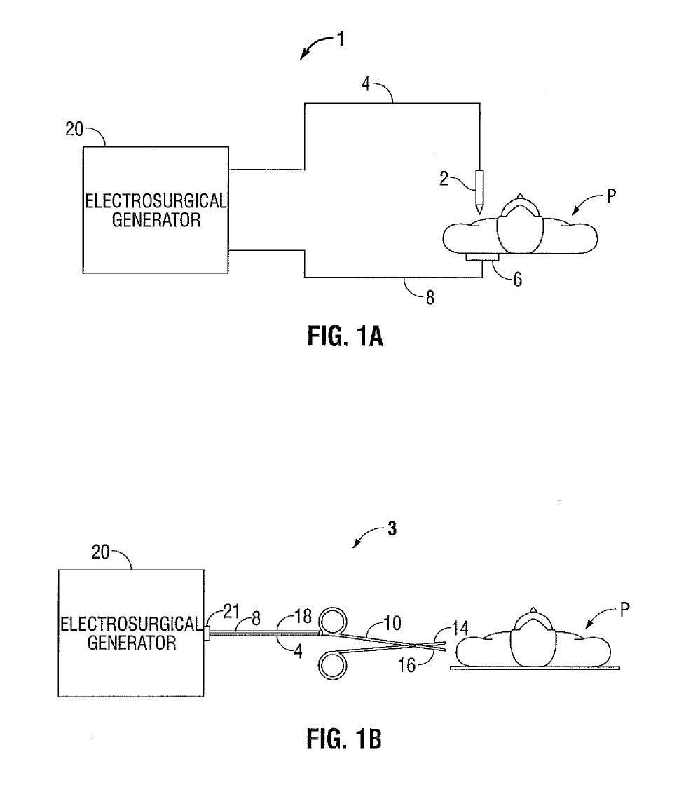

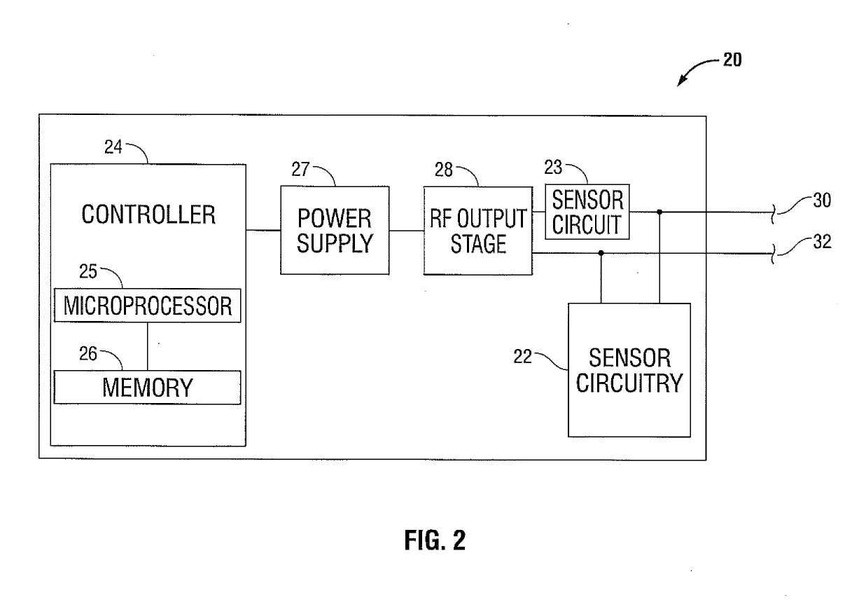

FIG. 1A is a schematic illustration of a monopolar electrosurgical system according to one embodiment of the present disclosure. The system includes an electrosurgical instrument 2 having one or more electrodes for treating tissue of a patient P. The instrument 2 is a monopolar type instrument including one or more active electrodes (e.g., electrosurgical cutting probe, ablation electrode(s), etc.). Electrosurgical RF energy is supplied to the instrument 2 by a generator 20 via a supply line 4, which is connected to an active terminal 30 (FIG. 2) of the generator 20, allowing the instrument 2 to coagulate, seal, ablate and/or otherwise treat tissue. The energy is returned to the generator 20 through a return electrode 6 via a return line 8 at a return terminal 32 (FIG. 2) of the generator 20. The active terminal 30 and the return terminal 32 are connectors configured to interface with plugs (not explicitly shown) disposed at the ends of the supply line 4 and the return line 8, respectively.

The system may include a plurality of return electrodes 6 that are arranged to minimize the chances of tissue damage by maximizing the overall contact area with the patient P. In addition, the generator 20 and the return electrode 6 may be configured for monitoring so-called “tissue-to-patient” contact to insure that sufficient contact exists therebetween to further minimize chances of tissue damage.

FIG. 1B is a schematic illustration of a bipolar electrosurgical system according to the present disclosure. The system includes a bipolar electrosurgical forceps 10 having one or more electrodes for treating tissue of a patient P. The electrosurgical forceps 10 includes opposing jaw members having an active electrode 14 and a return electrode 16 disposed therein. The active electrode 14 and the return electrode 16 are connected to the generator 20 through cable 18, which includes the supply and return lines 4, 8 coupled to the active and return terminals 30, 32, respectively (FIG. 2). The electrosurgical forceps 10 is coupled to the generator 20 at a connector 21 having connections to the active and return terminals 30 and 32 (e.g., pins) via a plug disposed at the end of the cable 18, wherein the plug includes contacts from the supply and return lines 4, 8.

The generator 20 includes suitable input controls (e.g., buttons, activators, switches, touch screen, etc.) for controlling the generator 20. In addition, the generator 20 may include one or more display screens for providing the user with variety of output information (e.g., intensity settings, treatment complete indicators, etc.). The controls allow the user to adjust power of the RF energy, waveform parameters (e.g., crest factor, duty cycle, etc.), and other parameters to achieve the desired waveform suitable for a particular task (e.g., coagulating, tissue sealing, intensity setting, etc.). The instrument 2 may also include a plurality of input controls that may be redundant with certain input controls of the generator 20. Placing the input controls at the instrument 2 allows for easier and faster modification of RF energy parameters during the surgical procedure without requiring interaction with the generator 20.

FIG. 2 shows a schematic block diagram of the generator 20 having a controller 24, a high voltage DC power supply 27 (“HVPS”) and an RF output stage 28. The HVPS 27 is connected to a conventional AC source (e.g., electrical wall outlet) and provides high voltage DC power to an RF output stage 28, which then converts high voltage DC power into RF energy and delivers the RF energy to the active terminal 30. The energy is returned thereto via the return terminal 32.

In particular, the RF output stage 28 generates waveforms (e.g., sinusoidal, square, or any type of AC waveform) of high RF energy. The RF output stage 28 is configured to generate a plurality of waveforms having various duty cycles, peak voltages, crest factors, and other suitable parameters. Certain types of waveforms are suitable for specific electrosurgical modes. For instance, the RF output stage 28 typically generates a 100% duty cycle sinusoidal waveform in cut mode, which is best suited for ablating, fusing and dissecting tissue and a 1-25% duty cycle waveform in coagulation mode, which is best used for cauterizing tissue to stop bleeding.

The generator 20 may include a plurality of connectors to accommodate various types of electrosurgical instruments (e.g., instrument 2, electrosurgical forceps 10, etc.). Further, the generator 20 is configured to operate in a variety of modes such as ablation, monopolar and bipolar cutting coagulation, etc. The generator 20 may include a switching mechanism (e.g., relays) to switch the supply of RF energy between the connectors, such that, for instance, when the instrument 2 is connected to the generator 20, only the monopolar plug receives RF energy.

The controller 24 includes a microprocessor 25 operably connected to a memory 26, which may be volatile type memory (e.g., RAM) and/or non-volatile type memory (e.g., flash media, disk media, etc.). The microprocessor 25 includes an output port that is operably connected to the HVPS 27 and/or RF output stage 28 allowing the microprocessor 25 to control the output of the generator 20 according to either open and/or closed control loop schemes. Those skilled in the art will appreciate that the microprocessor 25 may be substituted by any logic processor (e.g., control circuit) adapted to perform the calculations discussed herein.

A closed feedback control loop wherein sensor circuitry 22 and/or sensor circuitry 23, which may include a plurality of sensors for measuring a variety of tissue and energy properties (e.g., tissue impedance, tissue temperature, output current and/or voltage, voltage and current passing through the tissue, etc.), provides feedback to the controller 24. Such sensors are within the purview of those skilled in the art. The controller 24 signals the HVPS 27 and/or RF output stage 28, which then adjusts DC and/or RF power supply, respectively. The controller 24 also receives input signals from the input controls of the generator 20 or the instrument 2. The controller 24 utilizes the input signals to adjust power outputted by the generator 20 and/or performs other control functions thereon.

The present disclosure provides for a system and method for monitoring electrosurgical procedures using real power as opposed to reactive or imaginary power. The use of real power to control delivery of electrosurgical energy is discussed with respect to performing ablation procedures. Those skilled in the art will appreciate that the illustrated embodiments may be utilized with other electrosurgical procedures and/or modes.

Complex power consists of real and imaginary power components. Real power is identified with resistance or a purely resistive load and reactive or imaginary power is identified with reactance or a purely reactive load. Purely resistive impedance exhibits no phase shift between voltage and current, whereas reactance induces a phase shift θ between the voltage and the current passing through the tissue. More particularly, the phase shift θ is the angle by which the voltage phase leads the current phase.

Complex impedance consists of real and imaginary impedance. Real impedance is identified with resistance and imaginary impedance is identified with reactance. In addition, reactive impedance may be either inductive or capacitive. Purely resistive impedance exhibits no phase shift between the voltage and current, whereas reactance induces a phase shift θ between the voltage and the current passing through the tissue, thus imaginary impedance may be calculated based on the phase angle or phase shift between the voltage and current waveforms.

Electrosurgical generators sense various output parameters (e.g., voltage, current, etc.) within the generator. As such, both cables connected to the generator and patient tissue are sensed by the generator as electrical loads. These electrical loads are typically complex loads due to loop inductance, leakage capacitance, and complex tissue impedance. As a result, generator sensors do not accurately measure the tissue impedance but, rather, measure the total load associated with energy transmission. The amount of inaccuracy from this effect is dependent on the type of sensors employed and the differences between cable impedance and patient load impedance.

When attempting to control output power of an electrosurgical generator with the presence of the complex loads described above, as tissue resistance and total cable inductance change, the generator will adjust to the total impedance. This results in an inability of the generator to control tissue resistance and/or total cable inductance. A therapeutic effect requires that energy be delivered to the real component of the complex load since delivery of energy to the complex component results in stored energy in the reactive components rather than providing a direct therapeutic result. As a result, the energy delivered to the resistive component of the tissue will vary as the inductance of the cables vary, resulting in variable therapeutic outcomes.

The present disclosure provides for a method to address the above described variation in therapeutic effect by controlling the output of the generator based on the real component of the total load impedance and/or the real component of power delivered by the generator 20 to tissue. More specifically, the generator 20 obtains information (e.g., via a suitable sensor), such as complex impedance information for the cable and/or other suitable components, phase information related to the phase relationship between the current and voltage signals output by the generator 20, and information relating to the voltage and/or current output by the generator 20. The above stated information is used to calculate a real component of energy delivery that is, in turn, utilized to control generator 20 output.

The phase information describes a phase difference between current and voltage waveforms output by generator 20. In embodiments, the phase difference is determined by the microprocessor 25 by execution of a suitable software application, such as a single-band Fourier transform algorithm, a multi-band Fourier transform algorithm, an FFT algorithm, a Goertzel algorithm, an equivalent to a Fourier transform algorithm or a combination thereof. Commonly-owned U.S. Pat. No. 7,300,435, which is incorporated herein by reference in its entirety, describes a control system that uses a Goertzel algorithm to determine the phase difference between the voltage waveform and the current waveform output by an electrosurgical generator. The phase difference is used to determine the real component of the total load impedance.

The generator 20 is configured to increase or decrease output to ensure that the current delivered to the resistive component of the total load impedance maintains the desired output power. In this manner, the effect of inductance changes in cables and/or other suitable components on energy delivered to the resistive component of tissue is minimized or substantially reduced. This results in a consistent therapeutic effect and less sensitivity to variations in cable routing and/or placement.

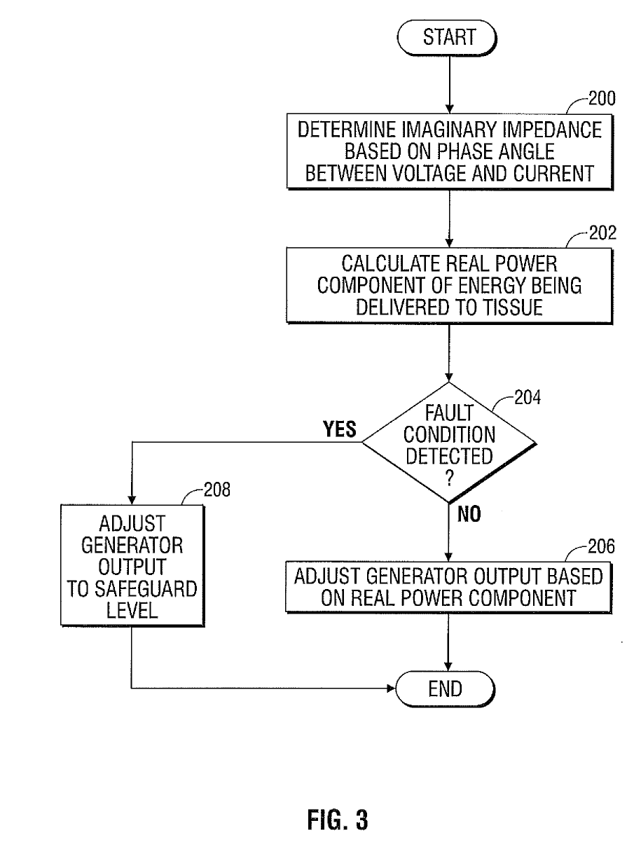

FIG. 3 shows a method for controlling output of the generator 20 based on the real power component of energy delivered by the generator 20 to tissue. The method may be embodied as a software application embedded in the memory 26 and executed by the microprocessor 25 to control generator 20 output.

With reference to FIG. 3, in step 200, ablation energy is delivered to tissue and the imaginary impedance is measured by the sensor circuitry 22 and/or the sensor circuitry 23. More specifically, sensor circuitry 22 and/or sensor circuitry 23 measures voltage and current waveforms passing through the tissue and determines the imaginary impedance (e.g., the imaginary component of the complex impedance) based on the phase angle between the waveforms.

In step 202, the real power component of the ablation energy being delivered by the generator 20 is calculated using the following formula (I):

P=VI cos θ (1)

In formula (I), P is the real power component, V is the voltage phase passing through tissue, I is the current phase passing through tissue, and θ is the angle of phase shift θ between the voltage phase and the current phase passing through the tissue. If tissue is purely resistive (i.e., a purely resistive load), the phase shift between voltage phase and current phase is 0° and, since, cos θ=1, P=VI if tissue is purely resistive.

Typically, total power output of the generator 20 exceeds the desired power delivered to tissue. This excess or reactive power is stored as potential energy in the reactive components (e.g., cables) of the system. Formula (1) takes into account the real power component of the ablation energy being delivered by the generator 20, effectively disregarding the excess or reactive power. The calculated real power component is then processed by the microprocessor 25 as input data.

In step 204, specific parameters are monitored by the microprocessor 25 to detect a possible fault condition. In one embodiment, the fault condition may be a predetermined maximum range between complex impedance and real impedance (i.e., a maximum imaginary or reactive impedance). Changes in the imaginary impedance during energy delivery may be used as an indication of changes in tissue properties due to energy application. More specifically, imaginary impedance may be used to detect the formation of microbubbles, bubble fields and tissue desiccation that impart an electrical reactivity to the tissue that corresponds to sensed imaginary impedance. The tissue reactivity is reflective of the energy that is being delivered into the tissue. Thus, the measured change in imaginary impedance or the measured change between complex impedance and real impedance may be used as an indication of the amount of energy resident in the tissue. Monitoring of the resident energy in combination with monitoring of the energy being supplied by the generator allows for calculation of energy escaping the tissue during treatment, thereby allowing for determination of efficiency of the treatment process as well as any inadvertent energy drains.

In embodiments, output of the generator 20 may also be controlled to a desired complex impedance to sustain microbubble formation. Changes in the complex impedance during energy delivery may be used as an indication of changes in tissue properties due to energy application. More specifically and as discussed above with reference to imaginary impedance, changes in complex impedance may also be used to detect the formation of microbubbles in that electrical reactivity imparted to tissue as a result of microbubble formation corresponds to changes in imaginary impedance. This change in imaginary impedance yields a corresponding change in complex impedance that may be monitored and used to control output of the generator 20.

In another embodiment, the fault condition may be a predetermined time limit that limits the amount of time for the generator 20 to achieve the desired power output level based on the real component of the total load impedance and/or the real component of power delivered by the generator 20 to tissue.

If the fault condition is detected in step 204, the fault condition is processed by the microprocessor 25 as input data and the controller 24 adjusts the output of the generator 20 to a pre-determined safeguard level in step 206. The safeguard level may be a discontinuance or termination of generator output or, alternatively, an output level pre-determined to be safe for the patient, the user, and/or components of the system should activation of generator 20 occur without a resistive load present or an inaccurate representation of real impedance be observed by the generator 20 due to a monitoring failure (e.g., sensor failure, microprocessor failure). Additionally or alternatively, generator output may be incrementally reduced in response to a detected fault condition such that for each incremental reduction in generator output, the microprocessor 25 re-checks for a possible fault condition. If the fault condition remains as detected by the microprocessor 25 following an increment of reduction, an additional increment of reduction is applied to generator output. If the fault condition is not detected by the microprocessor 25 following an increment of reduction, generator output is unchanged. In either case, the microprocessor 25 continues to monitor specific parameters for possible detection of a fault condition.

If the fault condition is not detected in step 204, in step 208 the real power component calculated in step 202 is processed by the microprocessor 25 as input data and the controller 24 adjusts the output of the generator 20 based on the processed real power component.

While several embodiments of the disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An electrosurgical generator for supplying electrosurgical energy to tissue, comprising:
sensor circuitry configured to measure a voltage phase and a current phase through tissue;
a processing device configured to compare the measured voltage and current phase to generate a real power component; and
a controller configured to regulate output of the electrosurgical generator based on at least one of the real power component and a predetermined imaginary impedance of tissue.

2. An electrosurgical generator according to claim 1, wherein the controller is configured to discontinue the output of the electrosurgical generator based on the predetermined imaginary impedance.

3. An electrosurgical generator according to claim 1, wherein the controller is configured to adjust the output of the electrosurgical generator to a predetermined level based on the predetermined imaginary impedance.

4. An electrosurgical generator according to claim 1, wherein the controller is configured to adjust the output of the electrosurgical generator based on an expiration of a predetermined period of time.

5. An electrosurgical generator according to claim 1, wherein the processing device is configured to calculate the real power component based on a difference between the measured voltage phase and current phase.

6. An electrosurgical generator according to claim 1, wherein the real power component corresponds to the output of the electrosurgical generator delivered to a resistive component of tissue.

7. A method for supplying electrosurgical energy to tissue, comprising the steps of:
measuring a voltage phase and a current phase through tissue;
comparing the measured voltage phase and the measured current phase to generate a real power component; and
regulating output of an electrosurgical generator based on at least one of the real power component and a predetermined range between a complex impedance of tissue and a real impedance of tissue.

8. A method according to claim 7, further comprising the step of:
calculating the real power component based on a difference between the measured voltage phase and current phase.

9. A method according to claim 7, further comprising the step of:
discontinuing the output of the electrosurgical generator based on the predetermined range between the complex and real tissue impedance.

10. A method according to claim 7, further comprising the step of:
adjusting the output of the electrosurgical generator to a predetermined level based on the predetermined range between the complex and real tissue impedance.

11. A method according to claim 7, further comprising the step of:
regulating the output of the electrosurgical generator based on an ability of the electrosurgical generator to output energy at a pre-determined level.

12. A method for supplying electrosurgical energy to tissue, comprising the steps of:
measuring a voltage phase and a current phase through tissue;
calculating a difference between the voltage phase and the current phase;
calculating a real power component of the electrosurgical energy based on at least one of the voltage phase, the current phase, and a difference between the voltage and current phases;
regulating output of the electrosurgical generator based on the real power component; and
adjusting the output of the electrosurgical generator to a pre-determined safeguard level based on a detected fault condition.

13. A method according to claim 12, wherein the detected fault condition is a predetermined imaginary impedance of tissue.

14. A method according to claim 12, further comprising the step of:
discontinuing the output of the electro surgical generator based on the detected fault condition.
